(19) United States
(12) Patent Application Publication
(10) Pub. No.: US 2015/0105679 A1
(43) Pub. Date: Apr. 16, 2015

(54) METHOD AND APPARATUS FOR GENERATING SYNCHRONIZATION SIGNAL USING ELECTROCARDIOGRAM SIGNAL

(71) Applicant: Electronics and Telecommunications Research Institute, Daejeon (KR)

(72) Inventor: Jung Hwan HWANG, Daejeon (KR)

(21) Appl. No.: 14/305,696

(22) Filed: Jun. 16, 2014

(30) Foreign Application Priority Data
Oct. 16, 2013 (KR) 10-2013-0123555

(51) Int. Cl. A61B 5/00; A61B 5/0402

(52) U.S. Cl. CPC A61B 5/7285; A61B 5/0402; A61B 5/0024
USPC 600/509

(57) ABSTRACT

An apparatus for generating a synchronization signal based on an electrocardiogram signal of a body by obtaining the electrocardiogram signal, outputting a first peak signal when strength of the electrocardiogram signal becomes larger than that of a reference signal, and generating a first synchronization signal depending on the first peak signal is provided.

### CROSS-REFERENCE TO RELATED APPLICATION

This application claims priority to and the benefit of Korean Patent Application No. 10-2013-0123555 filed in the Korean Intellectual Property Office on Oct. 16, 2013, the entire contents of which are incorporated herein by reference.

### BACKGROUND OF THE INVENTION

(a) Field of the Invention

The present invention relates to a method and an apparatus for generating a synchronization signal using an electrocardiogram signal of a body in an electronic communication device positioned at a region close to the body.

(b) Description of the Related Art

Recently, people have worn various electronic communication devices on their bodies or have carried electronic communication devices close to their bodies. For example, people have carried a smart phone for audio and data communication, and have captured photographs and videos through a head mounted device (HMD) such as smart goggles. Furthermore, in accordance with the development of wearable computer technology, the number and kinds of devices worn on a body have gradually increased.

Meanwhile, in order to monitor a health condition of a person in real time, various sensor devices such as a heart rate sensor and a fall sensor may be attached to a body and be used. In addition, an ultrasonic wave sensor is attached to a body of a blind person and is used to inform the blind person of an obstacle in front of them while walking.

These various electronic communication devices may be included in a body area network (BAN). In this case, the respective devices should be synchronized with each other. For example, since a sensor for monitoring health transmits collected health information to a portable terminal such as a smart phone, the portable terminal needs to restore received signals based on synchronization in order to recognize the health information. In addition, the ultrasonic wave sensor that may assist walking of the blind person may sense an obstacle in front of them by transmitting and receiving ultrasonic waves based on the synchronization.

According to the related art, a plurality of electronic communication devices (hereinafter referred to as “BAN devices”) included in the BAN have been synchronized with each other by transmitting and receiving a reference signal through a wired line, but have a limitation of using the wired line. In addition, a method for wirelessly transmitting the reference signal may be used. However, the wireless signal may be attenuated and lost while passing through the body and is sometimes not accurately transferred. A scheme of adding a separate signal to a data signal and then transmitting them rather than transmitting and receiving a separate reference signal may also be subjected to the above-mentioned problems. Further, in this scheme, the separate signal is further transmitted, and thus a data rate may be decreased.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a method and an apparatus for generating a synchronization signal of a body area network (BAN) device using an electrocardiogram signal of a body, for a wireless synchronization method that is not affected by attenuation of a signal by the body.

An exemplary embodiment of the present invention provides a method for generating a synchronization signal using an electrocardiogram signal of a body. The method includes: obtaining the electrocardiogram signal; outputting a first peak signal when strength of the electrocardiogram signal becomes higher than that of a reference signal; and generating a first synchronization signal depending on the first peak signal.

The obtaining may include amplifying the electrocardiogram signal and removing a noise signal from the amplified electrocardiogram signal.

The reference signal may be a signal always having constant strength, and the strength of the reference signal may be smaller than a minimum peak value of a QRS wave among waveforms included in the electrocardiogram signal and be smaller than a maximum peak value of another waveform rather than the QRS wave among the waveforms included in the electrocardiogram signal.

The method may further include: updating the reference signal; outputting a second peak signal when strength of the electrocardiogram signal becomes higher than that of the updated reference signal; and generating a second synchronization signal depending on the second peak signal.

The updating of the reference signal may include: measuring a plurality of time intervals at which a peak value of a QRS wave among waveforms included in the electrocardiogram signal appears; and comparing the plurality of measured time intervals with a predetermined time interval, respectively.

The comparing of the plurality of measured time intervals with the predetermined time interval may include: decreasing the reference signal in the case in which the time interval larger than the predetermined time interval is present, and measuring a time interval at which the peak value of the QRS wave appears using the decreased reference signal; and adjusting a ratio of a minimum value of the peak value to the reference signal in the case in which the time interval larger than the predetermined time interval is not present.

The method may further include removing a jitter signal from the first synchronization signal.

Another exemplary embodiment of the present invention provides an apparatus for generating a synchronization signal using an electrocardiogram signal of a body. The apparatus includes: an electrode unit obtaining the electrocardiogram signal; a signal comparer outputting a first peak signal when strength of the electrocardiogram signal becomes larger than that of a reference signal; and a synchronization signal generator generating a first synchronization signal depending on the first peak signal.

The apparatus may further include a filter removing a noise signal from the amplified electrocardiogram signal.

The reference signal may be a signal always having constant strength, and the strength of the reference signal may be smaller than a minimum peak value of a QRS wave among waveforms included in the electrocardiogram signal and may be -larger than a maximum peak value of another waveform rather than the QRS wave among the waveforms included in the electrocardiogram signal.

The apparatus may further include a signal processor updating the reference signal, wherein the signal comparer outputs a second peak signal when strength of the electrocardiogram signal becomes larger than that of the updated reference signal, and the synchronization signal generator generates a second synchronization signal depending on the second peak signal.

The signal processor may measure a plurality of time intervals at which a peak value of a QRS wave among waveforms included in the electrocardiogram signal appears, and compare the plurality of measured time intervals with a predetermined time interval, respectively.

The signal processor may decrease the reference signal in the case in which the time interval larger than the predetermined time interval is present and measure a time interval at which the peak value of the QRS wave appears using the decreased reference signal, and may adjust a ratio of a minimum value of the peak value to the reference signal in the case in which the time interval larger than the predetermined time interval is not present.

The signal processor may adjust a ratio of the reference signal based on minimum signal comparison performance of the signal comparer.

The apparatus may further include a jitter signal remover removing a jitter signal from the first synchronization signal.

As described above, according to an exemplary embodiment of the present invention, the plurality of BAN devices attached to a body of one person generate synchronization signals based on the electrocardiogram signals of the body, such that they may be individually synchronized with each other.

### BRIEF DESCRIPTION OF THE DRAWINGS

FIG. 1 is a drawing showing an apparatus 100 for generating a synchronization signal according to an exemplary embodiment of the present invention.

FIG. 2 is a graph showing various signals of the apparatus 100 for generating a synchronization signal according to an exemplary embodiment of the present invention.

FIG. 3 is a flowchart showing a process in which a signal processor of the apparatus 100 for generating a synchronization signal according to an exemplary embodiment of the present invention updates a reference signal.

FIG. 4 is a drawing showing signals transmitted and received by a plurality of BAN devices through a synchronization signal according to an exemplary embodiment of the present invention.

FIG. 5 is a drawing showing ultrasonic sensors synchronized with each other through a synchronization signal according to an exemplary embodiment of the present invention.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

Throughout the specification, unless explicitly described to the contrary, the word “comprise” and variations such as “comprises” or “comprising” will be understood to imply the inclusion of stated elements but not the exclusion of any other elements. In addition, the terms “-er”, “-or”, and “module” described in the specification mean units for processing at least one function and operation, and can be implemented by hardware components or software components and combinations thereof.

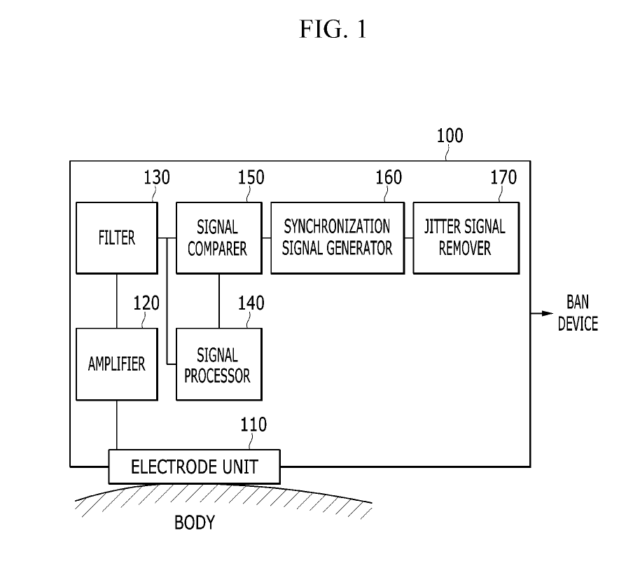

FIG. 1 is a drawing showing an apparatus 100 for generating a synchronization signal according to an exemplary embodiment of the present invention.

Referring to FIG. 1, an apparatus 100 for generating a synchronization signal according to an exemplary embodiment of the present invention is configured to include an electrode unit 110, an amplifier 120, a filter 130, a signal processor 140, a signal comparer 150, a synchronization signal generator 160, and a jitter signal remover 170.

The electrode unit 110 obtains electrocardiogram signals through a plurality of electrodes obtaining electrocardiogram signals from a body. That is, the plurality of electrodes included in the electrode unit 110 are attached to a surface of the body, and detect a potential formed on skin by an active current generated from the myocardium. Therefore, the electrocardiogram signal obtained by the electrode unit 110 has a potential difference form. Magnitudes of the electrocardiogram signals may be different from each other depending on positions that the electrode unit contacts. However, shapes of the electrocardiogram signals are the same as each other, and delay times of the electrocardiogram signals depending on measurement positions are very low.

The amplifier 120 amplifies the electrocardiogram signals obtained by the electrode unit 110. That is, the amplifier 120 combines the electrocardiogram signals detected by the plurality of electrodes and having the potential difference forms, and amplifies the electrocardiogram signals.

The filter 130 removes noise signals from the amplified electrocardiogram signals. That is, the filter 130 removes noise signals caused by various bioelectric currents from the electrocardiogram signals.

The signal processor 140 generates a reference signal required for generating a synchronization signal using the electrocardiogram signal from which the noise signal is removed. In addition, the signal processor 140 may update the reference signal depending on a control signal for the reference signal received from a body area network (BAN) device.

The signal comparer 150 compares the electrocardiogram signal from which the noise signal is removed with the reference signal generated by the signal processor 140, and outputs a peak signal required for generating the synchronization signal. In this case, the signal comparer 150 detects a peak value of the electrocardiogram signal in a section in which strength of the electrocardiogram signal is higher than that of the reference signal, and outputs the peak signal.

The synchronization signal generator 160 generates the synchronization signal in accordance with the peak signal output from the signal comparer 150. Here, the generated synchronization signal has a predetermined pulse width.

The jitter signal remover 170 removes a jitter signal from the generated synchronization signal. Since a phase noise signal is present in the electrocardiogram signal detected by the electrode, the jitter signal may be included in the synchronization signal. The jitter signal included in the synchronization signal is removed by the jitter signal remover 170.

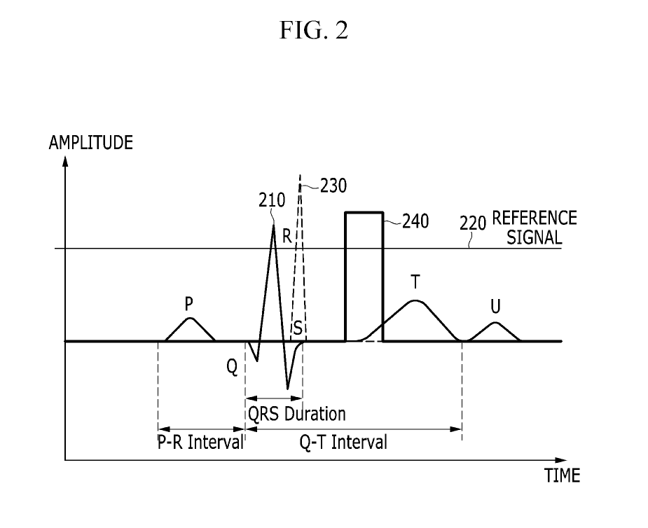

FIG. 2 is a graph showing various signals of the apparatus 100 for generating a synchronization signal according to an exemplary embodiment of the present invention.

The apparatus 100 for generating a synchronization signal according to an exemplary embodiment of the present invention compares the electrocardiogram signal 210 output from the filter 130 with the reference signal 220, and generates the output peak signal 230.

The electrocardiogram signal 210 from which the noise signal is removed by the filter 130 has specific waveforms such as a P wave, a QRS wave, and the like, wherein the specific waveforms are associated with contraction and dilatation movement of a heart. The QRS wave among the waveforms included in the electrocardiogram signal 210 shows the highest amplitude, and the apparatus 100 for generating a synchronization signal according to an exemplary embodiment of the present invention may generate the synchronization signal 240 using the QRS wave. That is, the signal comparer 150 compares the electrocardiogram signal 210 with the reference signal 220 to determine a section in which the electrocardiogram signal 210 is larger than the reference signal 220 (hereinafter referred to as a “QRS section”). Therefore, the reference signal 220 is a signal always having constant strength, and the strength of the reference signal 220 should be smaller than a minimum peak value of the QRS wave and be larger than a maximum peak value of another signal included in the electrocardiogram signal 210.

The signal comparer 150 detects a peak value in the QRS section and outputs the peak signal 230. Here, the peak signal 230 output from the signal comparer 150 is delayed as compared with a time at which the peak value of the QRS waveform actually appears due to a time delay in the signal processor 140.

Then, the synchronization signal generator 160 outputs the synchronization signal 240 having a predetermined pulse width in accordance with a time corresponding to the peak of the output peak signal 230. The synchronization signal 240 is delayed as compared with the peak signal 230 due to a time delay in the synchronization signal generator 160, similar to the peak signal 230.

Here, forms of the peak signal 230 and the synchronization signal 240 according to an exemplary embodiment of the present invention shown in FIG. 2 are illustrative. That is, all forms satisfying an object of each signal may be used.

In addition, amplitudes of the QRS waveforms may be different for every person and may be changed over time. Therefore, the reference signal 220 used in order to distinguish between the QRS waveforms should be updated per a predetermined period, and is updated by the signal processor 140. Meanwhile, while the signal processor 140 updates the reference signal 220, the apparatus 100 for generating a synchronization signal continuously generates the synchronization signal 240 using an existing reference signal 220.

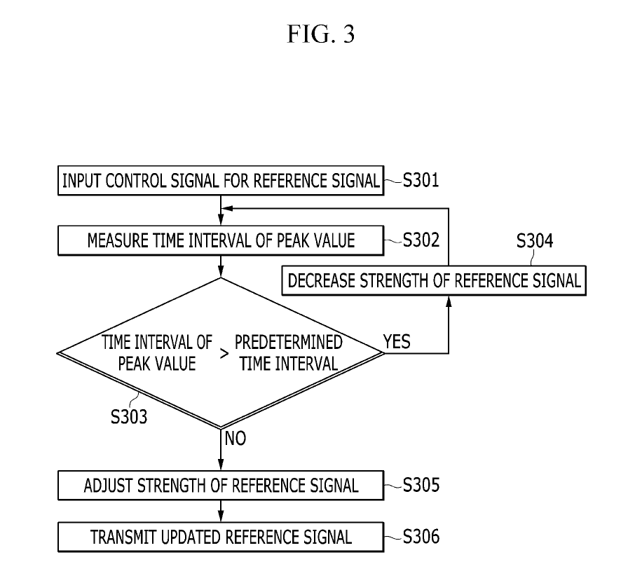

FIG. 3 is a flowchart showing a process in which a signal processor of the apparatus 100 for generating a synchronization signal according to an exemplary embodiment of the present invention updates a reference signal.

First, the control signal for the reference signal 220 is input from the BAN device to the signal processor 140 per a predetermined period (S301). Then, the signal processor 140 compares the electrocardiogram signal 210 output from the filter 130 with the reference signal 220 for the predetermined period, and measures a time interval at which the peak value of the QRS waveform appears (S302).

Next, the signal processor 140 compares the time interval at which the peak value appears with a predetermined time interval (S303). The predetermined period may be determined depending on importance of synchronization. That is, a BAN device of which synchronization is important frequently updates the reference signal 220, thereby making it possible to minimize a synchronization error, and the predetermined period is extended, thereby making it possible to more accurately obtain the time interval of the peak value.

The signal processor 140 decreases the reference signal 220 (S304) when a time interval of the peak value larger than the predetermined time interval is present. That is, in this case, the signal processor 140 determines that the existing reference signal 220 is excessively high, such that it is not appropriate for detecting the QRS waveform. Therefore, the signal processor 140 decreases a magnitude of the reference signal 220 so that the peak value of the QRS waveform may be detected, and again measures the time interval of the peak value through the decreased reference signal 220. Meanwhile, the predetermined time interval may be determined in consideration of a rhythm of an electrocardiogram of a general person. That is, when it is assumed that a rhythm of an electrocardiogram of a general person is 100 per minute, a time interval of a peak value may be 1/100 [min], and a predetermined time interval may be 1/90 to 1/80 [min] slightly larger than 1/100 [min].

Then, when all of the time intervals of the peak values measured for the predetermined period become smaller than the predetermined time interval, a ratio of a minimum peak value, which has the smallest strength among the peak values, to the reference signal 220 is adjusted (S305). That is, a magnitude of the reference signal 220 is adjusted to set a ratio of the minimum peak value to the reference signal 220 to a fixed ratio, such that the signal comparer may stably compare the peak value with the reference signal 220. The fixed ratio may be predetermined based on minimum signal comparison performance of the signal comparer.

Then, the signal processor 140 may transmit the updated reference signal 220 to the signal comparer 150 to allow a new synchronization signal 240 to be generated (S306). In addition, the signal processor transmits the control signal for the reference signal 220 to the BAN device connected to the apparatus 100 for generating a synchronization signal to inform the BAN device that the reference signal 220 has been updated.

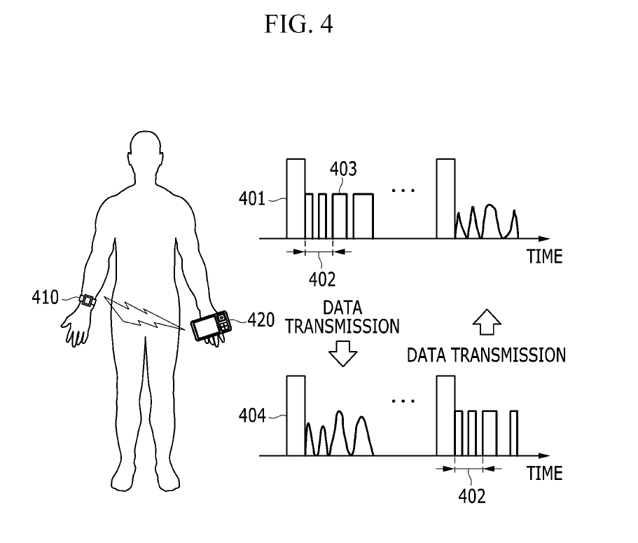

FIG. 4 is a drawing showing signals transmitted and received by a plurality of BAN devices through a synchronization signal according to an exemplary embodiment of the present invention.

Devices close to a body of a user are synchronized with each other through the synchronization signals generated by the apparatuses 100 for generating a synchronization signal according to an exemplary embodiment of the present invention. In this case, the apparatuses 100 for generating a synchronization signal may be embedded in the respective BAN devices or be connected to the BAN devices outside the BAN devices. In the case in which the apparatuses 100 for generating a synchronization signal are embedded in the respective BAN devices, the electrode units 110 of the apparatuses 100 for generating a synchronization signal may be exposed to the outside to obtain the electrocardiogram signals 210 of the body.

The respective BAN devices may generate the synchronization signals based on the electrocardiogram signals 210 of the body using the apparatuses 100 for generating a synchronization signal disposed at an inner portion or an outer portion thereof. The synchronization signals generated by the respective apparatuses 100 for generating a synchronization signal may have the same waveform and start time. Then, the respective BAN devices may transmit and receive data to and from other BAN devices using the synchronization signals.

Referring to FIG. 4, in the case in which a first BAN device 410 transmits data to a second BAN device 420, it transmits a data signal 403 after a predetermined time 402 elapses from a synchronization signal 401. That is, a point in time at which the data signal 403 is transmitted is determined based on the synchronization signal 401, and the predetermined times are the same in all BAN devices synchronized with each other by synchronization signals 401 and 404 generated through the electrocardiogram signal 210 of one person. An existing wired or wireless communication scheme may be used as it is as a scheme for transmitting and receiving the data signal in the respective BAN devices 410 and 420, and a form of the data signal may be changed depending on a communication scheme.

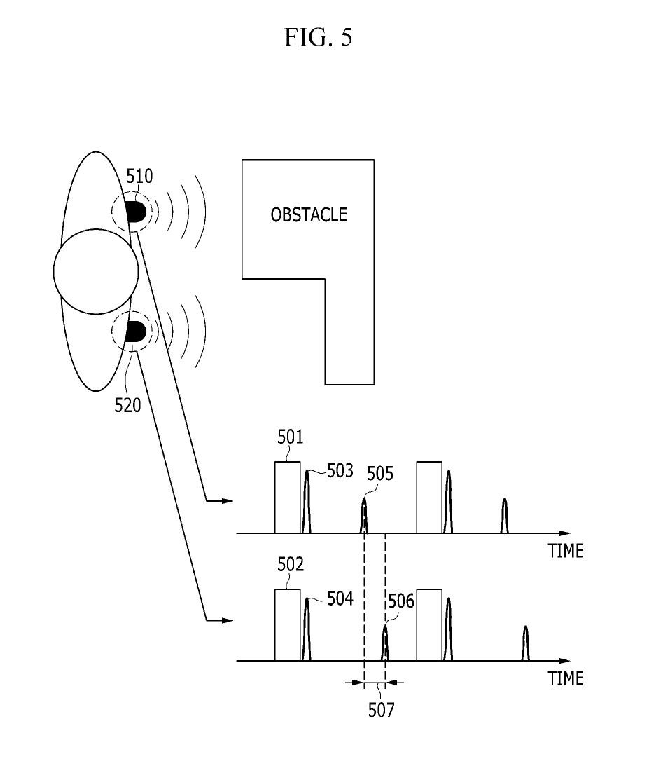

FIG. 5 is a drawing showing ultrasonic sensors synchronized with each other through a synchronization signal according to an exemplary embodiment of the present invention.

An ultrasonic wave sensor included in the BAN is used to detect an obstacle in front of a blind person, thereby making it possible to assist walking of the blind person. That is, the ultrasonic wave sensor radiates an ultrasonic wave forward and receives an ultrasonic wave signal reflected on the obstacle and returning thereto, thereby making it possible to inform the blind person that the obstacle is present in front of them. However, in the case in which only one ultrasonic wave sensor is used, only whether or not the obstacle is present may be detected, and it is impossible to obtain a form of the obstacle.

A plurality of ultrasonic wave sensors 510 and 520 included in the BAN according to an exemplary embodiment of the present invention may be synchronized with each other using synchronization signals 501 and 502 generated through the electrocardiogram signal 210, and they compare times required for radiated ultrasonic wave signals 503 and 504 to return thereto with each other to obtain the form of the obstacle.

The plurality of ultrasonic wave sensors 510 and 520 close to a body of the blind person are synchronized with each other through the synchronization signals 501 and 502 generated by the apparatuses 100 for generating a synchronization signal disposed at an inner portion or an outer portion thereof. The ultrasonic wave sensors 510 and 520 simultaneously radiate the ultrasonic waves 503 and 504 forward, respectively, after a predetermined time elapses from the synchronization signals 501 and 502, and receive reflected ultrasonic waves 505 and 506, respectively. In this case, since all of the distances from the body to the obstacles are different from each other, the reflected ultrasonic waves may be received at different times 507, and all different distances from the respective ultrasonic wave sensors to the obstacle may be calculated. That is, when the distances calculated through a time at which the reflected ultrasonic waves are received are connected to each other, a form of the obstacle positioned in front of the blind person may be recognized.

Referring to FIG. 5, since a time at which a first ultrasonic wave sensor 510 receives the reflected ultrasonic wave is ahead of a time at which a second ultrasonic wave sensor 520 receives the reflected ultrasonic wave, it may be recognized that a distance from a place at which the first ultrasonic wave sensor 510 is positioned to the obstacle is shorter than a distance from a place at which the second ultrasonic wave sensor 520 is positioned to the obstacle. In the case in which n ultrasonic wave sensors are used, when all of the distances from first to n-th ultrasonic wave sensors to the obstacle are connected to each other, a form of the obstacle may be recognized.

As described above, according to an exemplary embodiment of the present invention, the plurality of BAN devices attached to a body of one person generate the synchronization signals based on the electrocardiogram signals of the body, such that they may be individually synchronized with each other.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A method for generating a synchronization signal using an electrocardiogram signal of a body, comprising:
obtaining the electrocardiogram signal;
outputting a first peak signal when strength of the electrocardiogram signal becomes higher than that of a reference signal; and
generating a first synchronization signal depending on the first peak signal.

2. The method of claim 1, wherein
the obtaining includes
amplifying the electrocardiogram signal and removing a noise signal from the amplified electrocardiogram signal.

3. The method of claim 1, wherein
the reference signal is a signal having constant strength, and the strength of the reference signal is smaller than a minimum peak value of a QRS wave among waveforms included in the electrocardiogram signal and is larger than a maximum peak value of another waveform rather than the QRS wave among the waveforms included in the electrocardiogram signal.

4. The method of claim 1, further comprising:
updating the reference signal;
outputting a second peak signal when strength of the electrocardiogram signal becomes higher than that of the updated reference signal; and
generating a second synchronization signal depending on the second peak signal.

5. The method of claim 4, wherein
the updating of the reference signal includes:
measuring a plurality of time intervals at which a peak value of a QRS wave among waveforms included in the electrocardiogram signal appears; and
comparing the plurality of measured time intervals with a predetermined time interval, respectively.

6. The method of claim 5, wherein
the comparing of the plurality of measured time intervals with the predetermined time interval includes:
decreasing the reference signal in the case in which the time interval larger than the predetermined time interval is present, and measuring a time interval at which the peak value of the QRS wave appears using the decreased reference signal; and
adjusting a ratio of a minimum value of the peak value to the reference signal in the case in which the time interval larger than the predetermined time interval is not present.

7. The method of claim 1, further comprising
removing a jitter signal from the first synchronization signal.

8. An apparatus for generating a synchronization signal using an electrocardiogram signal of a body, comprising:
an electrode unit obtaining the electrocardiogram signal;
a signal comparer outputting a first peak signal when strength of the electrocardiogram signal becomes higher than that of a reference signal; and
a synchronization signal generator generating a first synchronization signal depending on the first peak signal.

9. The apparatus of claim 8, further comprising:
an amplifier amplifying the electrocardiogram signal; and
a filter removing a noise signal from the amplified electrocardiogram signal.

10. The apparatus of claim 8, wherein
the reference signal is a signal having constant strength, and the strength of the reference signal is smaller than a minimum peak value of a QRS wave among waveforms included in the electrocardiogram signal and is larger than a maximum peak value of another waveform rather than the QRS wave among the waveforms included in the electrocardiogram signal.

11. The apparatus of claim 8, further comprising
a signal processor updating the reference signal,
wherein the signal comparer
outputs a second peak signal when strength of the electrocardiogram signal becomes higher than that of the updated reference signal, and
the synchronization signal generator
generates a second synchronization signal depending on the second peak signal.

12. The apparatus of claim 11, wherein
the signal processor
measures a plurality of time intervals at which a peak value of a QRS wave among waveforms included in the electrocardiogram signal appears, and compares the plurality of measured time intervals with a predetermined time interval, respectively.

13. The apparatus of claim 12, wherein
the signal processor
decreases the reference signal in a case in which the time interval larger than the predetermined time interval is present and measures a time interval at which the peak value of the QRS wave appears using the decreased reference signal, and
adjusts a ratio of a minimum value of the peak value to the reference signal in the case in which the time interval larger than the predetermined time interval is not present.

14. The apparatus of claim 13, wherein
the signal processor
adjusts a ratio of the reference signal based on minimum signal comparison performance of the signal comparer.

15. The apparatus of claim 8, further comprising
a jitter signal remover removing a jitter signal from the first synchronization signal.
